(19) United States
(12) Patent Application Publication
(10) Pub. No.: US 2022/0346718 A1
(43) Pub. Date: Nov. 3, 2022

(54) BIOLOGICAL INFORMATION MEASURING DEVICE

(71) Applicant: OMRON HEALTHCARE Co., Ltd., Kyoto (JP)

(72) Inventor: Kenji ONO, Kyoto (JP)

(21) Appl. No.: 17/810,754

(22) Filed: Jul. 5, 2022

Related U.S. Application Data
(63) Continuation of application No. PCT/JP2021/000146, filed on Jan. 6, 2021.

(30) Foreign Application Priority Data
Jan. 10, 2020 (JP) 2020-003051

(51) Int. Cl. A61B 5/00; A61B 5/282; A61B 5/308; A61B 5/332

(52) U.S. Cl. CPC A61B 5/6843; A61B 5/282; A61B 5/308; A61B 5/332

(57) ABSTRACT

A first, second and third electrode, an electrode contact detection means configured to detect and output a state in which all of the electrodes are in contact with a surface of a measurement target, and control means configured to execute a measurement process of biological information, wherein the electrode contact detection means includes a bias power source configured to apply a voltage to each of the first electrode and the second electrode, a first comparator and a second comparator, and a contact state determination unit configured to determine whether all of the electrodes are in contact with the surface of the measurement target based on outputs of the first comparator and the second comparator, and the control means is configured to execute a process for opening the bias power source and executes the measurement process only when all electrodes are in contact with the surface of the measurement target.

### CROSS-REFERENCE TO RELATED APPLICATIONS

This application is the U.S. national stage application filed pursuant to 35 U.S.C. 365(c) and 120 as a continuation of International Patent Application No. PCT/JP2021/000146, filed Jan. 6, 2021, which application claims priority to Japanese Patent Application No. 2020-003051, filed Jan. 10, 2020, which applications are incorporated herein by reference in their entireties.

### FIELD

The present invention belongs to the technical field related to healthcare, and particularly relates to a biological information measurement device.

### BACKGROUND

In recent years, it has become widespread to perform health management by: measuring information (hereinafter, also referred to as biological information) on the body and health of an individual such as a blood pressure value and an electrocardiographic waveform with a measurement device; and recording and analyzing the measurement result with an information terminal.

As an example of the measurement device as described above, a portable electrocardiographic measurement device configured to measure an electrocardiographic waveform immediately when an abnormality occurs in everyday life, such as pain and palpitation in a chest, has been proposed, and an early detection of heart disease or a contribution to appropriate treatment is expected (for example, JP H9-56686 A).

JP H9-56686 A discloses a portable electrocardiograph including three electrodes for measurement in a main body, and the document proposes a technique for obtaining an accurate electrocardiographic signal by preventing baseline fluctuation of the electrocardiographic signal due to a change in pressure of a hand holding the main body. Specifically, it is described that a third measurement electrode using a part of a hand holding the electrocardiograph as a reference potential is provided, and a difference between a potential difference between the third measurement electrode and a first measurement electrode brought into contact with the chest and a potential difference between the third measurement electrode and a second measurement electrode brought into contact with the holding hand is amplified as an electrocardiographic signal.

### SUMMARY OF INVENTION

### Technical Problem

However, even with the technique described in JP H9-56686 A, when measurement is performed in a state in which the three electrodes are not correctly in contact with the measurement target, the contact resistance between the electrodes and (the skin of) the measurement target does not become sufficiently low, and consequently, there is a problem in that accurate measurement of biological information cannot be performed.

In view of the conventional technology described above, an object of the present invention is to provide a technique capable of executing measurement only when all of three electrodes are appropriately in contact with a measurement target and measuring biological information with high accuracy in a biological information measurement device using three or more electrodes.

### Solution to Problem

In order to solve the above problems, the biological information measurement device according to the present invention includes a first electrode, a second electrode, and a third electrode, the biological information measurement device measuring biological information of a measurement target based on a potential difference between the first electrode and the second electrode, the biological information measurement device including: an electrode contact detection means configured to detect and output a state in which all of the first electrode, the second electrode, and the third electrode are in contact with a surface of the measurement target; and a control means configured to execute a measurement process of measuring the biological information, wherein the electrode contact detection means includes a bias power source configured to apply a voltage to each of the first electrode and the second electrode so that the first electrode and the second electrode have a contact detection potential higher than a potential of the third electrode; a first comparator and a second comparator connected to the first electrode and the second electrode, respectively, and configured to compare the contact detection potential with respective potentials of the first electrode and the second electrode; and a contact state determination unit configured to determine whether or not all of the first electrode, the second electrode, and the third electrode are in contact with the surface of the measurement target based on outputs of the first comparator and the second comparator, and the control means is configured to execute a process of opening the first electrode, the second electrode, and the bias power source, and execute the measurement process when the electrode contact detection means outputs that all of the first electrode, the second electrode, and the third electrode are in contact with the surface of the measurement target.

Here, the bias power source may be a common power source for the first electrode and the second electrode, or may be a separate power source for each electrode.

With the above-described configuration, measurement is not started unless all of the three electrodes appropriately come into contact with the surface of the measurement target, and thus biological information can appropriately be measured with a signal having a high S (Signal)/N (Noise) ratio. In addition, since the control means executes a process of turning OFF the bias power source from the circuit before performing the measurement process, noise generated due to connection of the power source can be eliminated.

Further the third electrode may be a ground electrode, the biological information measurement device may include a first differential amplifier connected to the first electrode and the second electrode, the first differential amplifier being configured to amplify and output a potential difference between the first electrode and the second electrode, and the control means may be configured to measure the biological information of the measurement target based on an output of the first differential amplifier.

With such a configuration, a ground (GND) can commonly be used with an AD (Analog to Digital) conversion unit for a signal, and it becomes easy to remove an in-phase noise of the signal at the time of AD conversion.

Further, a second differential amplifier connected to the first electrode and the third electrode, and configured to amplify and output a potential difference between the first electrode and the third electrode; a third differential amplifier connected to the second electrode and the third electrode, and configured to amplify and output a potential difference between the second electrode and the third electrode; and a fourth differential amplifier connected to output sides of the second differential amplifier and the third differential amplifier, and configured to amplify and output a potential difference between an output voltage of the second differential amplifier and an output voltage of the third differential amplifier may be provided, wherein the control means may be configured to measure the biological information of the measurement target based on an output of the fourth differential amplifier.

With such a configuration, when the analog signal output from the fourth differential amplifier is amplified, the in-phase noise of the signal can be easily removed.

Further, the biological information may be an electrocardiographic waveform, that is, the biological information measurement device may be an electrocardiograph. In the measurement of an electrocardiographic waveform, it is necessary to measure a more delicate change in a signal, and therefore, the present invention capable of obtaining a signal with less noise and high accuracy is suitably applied.

### Advantageous Effects of Invention

According to the present invention, a technique capable of executing measurement only when all of three electrodes are appropriately in contact with a measurement target and measuring biological information with high accuracy can be provided in a biological information measurement device using three or more electrodes.

### BRIEF DESCRIPTION OF DRAWINGS

FIG. 1(A) is a front view illustrating the configuration of the portable electrocardiographic measurement device according to the embodiment;

FIG. 1(B) is a rear view illustrating the configuration of the portable electrocardiographic measurement device according to the embodiment;

FIG. 1(C) is a left side view illustrating the configuration of the portable electrocardiographic measurement device according to the embodiment;

FIG. 1(D) is a right side view illustrating the configuration of the portable electrocardiographic measurement device according to the embodiment;

FIG. 1(E) is a plan view illustrating the configuration of the portable electrocardiographic measurement device according to the embodiment;

FIG. 1(F) is a bottom view illustrating the configuration of the portable electrocardiographic measurement device according to the embodiment;

FIG. 2 is a block diagram illustrating a functional configuration of the portable electrocardiographic measurement device according to the embodiment;

FIG. 3 is a circuit diagram illustrating a part of an electric circuit configuration of the portable electrocardiographic measurement device according to a first embodiment;

FIG. 4 is a flowchart illustrating a flow of electrocardiographic waveform measurement processing in the portable electrocardiographic measurement device according to the embodiment;

FIG. 5 is a flowchart illustrating a subroutine for performing electrode contact detection processing in the portable electrocardiographic measurement device according to the embodiment; and,

FIG. 6 is a circuit diagram illustrating a part of an electric circuit configuration of a portable electrocardiographic measurement device according to a modification example.

### DESCRIPTION OF EMBODIMENTS

### First Embodiment

Embodiments of the present invention will be specifically described below with reference to the drawings. It should be noted that the dimension, material, shape, relative arrangement and the like of the components described in the present embodiment are not intended to limit the scope of this invention to them alone, unless otherwise stated.

### Electrocardiographic Measurement Device

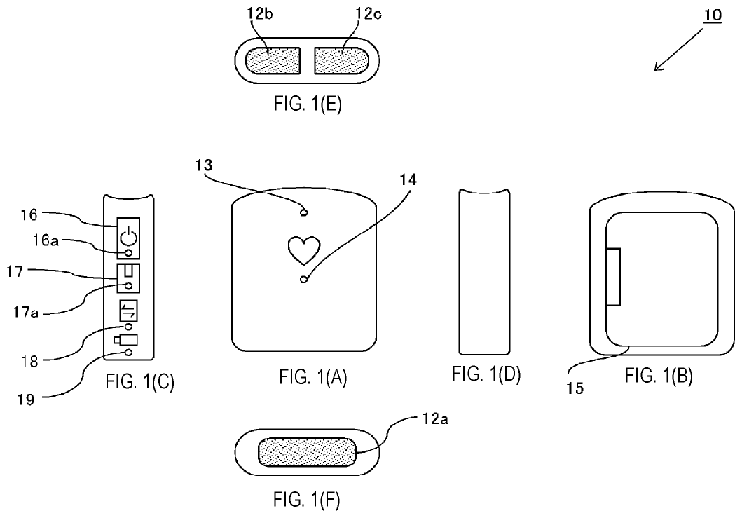

FIGS. 1(A) through (F) generally illustrate a configuration of a portable electrocardiograph 10 according to the present embodiment. FIG. 1(A) is a front view illustrating the front of the body. Similarly, FIG. 1(B) is a rear view, FIG. 1(C) is a left side view, FIG. 1(D) is a right side view, FIG. 1(E) is a plan view, and FIG. 1(F) is a bottom view.

A bottom surface of the portable electrocardiograph 10 is provided with a left electrode 12a brought into contact with the left side of the body during electrocardiographic measurement. A top surface side of the portable electrocardiograph 10, opposite to the bottom surface, is provided with a first right electrode 12b similarly brought into contact with the center of the right-hand index finger and a second right electrode 12c brought into contact with the base of the right-hand index finger.

During electrocardiographic measurement, the portable electrocardiograph 10 is held by the right hand, and the right-hand index finger is placed on the top surface portion of the portable electrocardiograph 10 in proper contact with the first right electrode 12b and the second right electrode 12c. The left electrode is then brought into contact with the skin at a location corresponding to the desired measurement. For example, when measurement is performed by the so-called lead I, the left electrode is brought into contact with the palm of the left hand, and when measurement is performed by the so-called V4 lead, the left electrode is brought into contact with the skin slightly to the left of the epigastric region of the left chest and below the papilla.

In addition, various operation units and indicators are disposed on a left side surface of the portable electrocardiograph 10. Specifically, a power switch 16, a power source LED 16a, a Bluetooth (registered trademark) Low Energy (BLE) communication button 17, a BLE communication LED 17a, a memory residual display LED 18, a battery exchange LED 19, and the like, are provided.

Additionally, a measurement state notification LED 13, an analysis result notification LED 14, and the like are provided at the front surface of the portable electrocardiograph 10, and a battery housing opening and a battery cover 15 are arranged at the rear surface of the portable electrocardiograph 10.

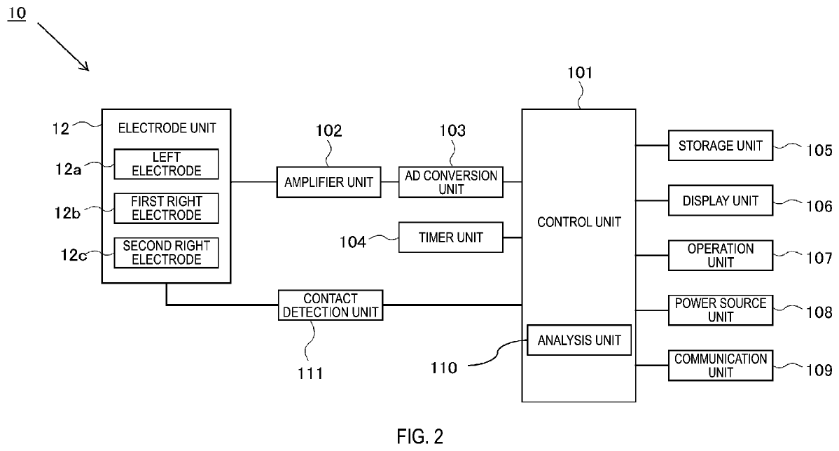

Also, in FIG. 2, a block diagram illustrating a functional configuration of the portable electrocardiograph 10 is described. As illustrated in FIG. 2, the portable electrocardiograph 10 includes function units including a control unit 101, an electrode unit 12, an amplifier unit 102, an AD conversion unit 103, a timer unit 104, a storage unit 105, a display unit 106, an operation unit 107, a power source unit 108, a communication unit 109, an analysis unit 110, and a contact detection unit 111.

The control unit 101 manages the control of the portable electrocardiograph 10, and includes a central processing unit (CPU) and the like, for example. In response to receiving operation of the user via the operation unit 107, the control unit 101 controls each component of the portable electrocardiograph 10 to execute various processing operations such as electrocardiographic measurement and information communication in accordance with a predetermined program. Note that the predetermined program is stored in the storage unit 105 described below.

Additionally, the control unit 101 includes, as a functional module, the analysis unit 110 analyzing electrocardiographic waveforms. The analysis unit 110 analyzes the measured electrocardiographic waveform for the presence of disturbance or the like, and outputs a result indicating whether the electrocardiographic waveform obtained at least during measurement is normal.

The electrode unit 12 includes the left electrode 12a, the first right electrode 12b, and the second right electrode 12c, and functions as a sensor for detecting an electrocardiographic waveform. The amplifier unit 102 has a function of amplifying a signal indicating an electrocardiographic waveform output from the electrode unit 12 as described later. The AD conversion unit 103 functions to convert an analog signal amplified by the amplifier unit 102 into a digital signal and to transmit the converted signal to the control unit 101.

The timer unit 104 has a function of measuring time with reference to the RTC (Real Time Clock). For example, as will be described later, when the electrode contact detection process is performed, the time during which all of the left electrode 12a, the first right electrode 12b, and the second right electrode 12c are in contact with the body is counted. Further, the period of time until the end of measurement may be counted and output during the electrocardiographic measurement.

The storage unit 105 includes a main storage device such as a random access memory (RAM), and stores various kinds of information such as an application program, a measured electrocardiographic waveform, and an analysis result. In addition to the RAM, for example, a long-term storage medium such as a flash memory may be provided.

The display unit 106 is configured to include the power source LED 16a, the BLE communication LED 17a, the memory residual display LED 18, the battery exchange LED 19, and the like described above, and transmits the state of the device to the user by turning on or blinking the LED. Furthermore, the operation unit 107 includes the power switch 16, the communication button 17, and the like, and receives input operation from a user, and has a function for causing the control unit 101 to execute a process in response to the operation.

The power source unit 108 is configured to include a battery that supplies the power required for operation of the device. The battery may be, for example, a secondary battery such as a lithium ion battery, or a primary battery.

The communication unit 109 includes an antenna for wireless communication, and has a function of communicating with another device such as an information processing terminal by at least BLE communication. Additionally, a terminal may be provided for wired communication.

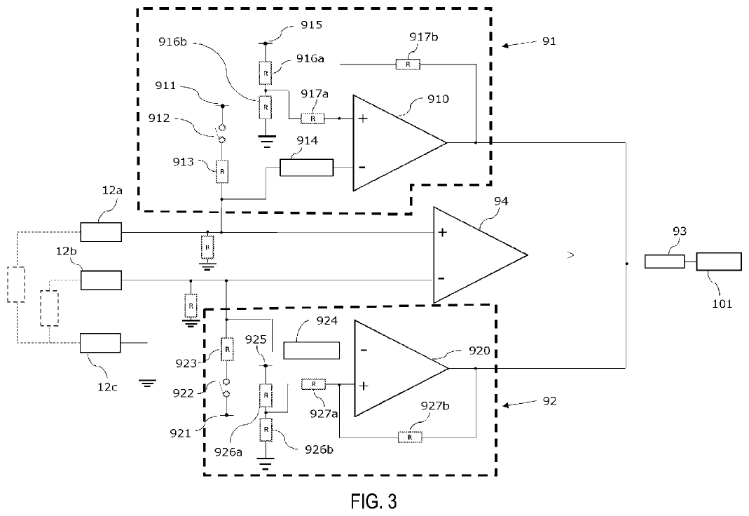

The contact detection unit 111 is configured to include an electric circuit connected to the left electrode 12a and the first right electrode 12b, and has a function of detecting and outputting a state in which all of the left electrode 12a, the first right electrode 12b, and the second right electrode 12c are correctly in contact with the respective parts of the body. The contact detection unit 111 is described in detail below on the basis of FIG. 3. FIG. 3 is a circuit diagram illustrating an electrical circuit constituting the contact detection unit 111.

The contact detection unit 111 generally includes a left detection unit 91 connected to the left electrode 12a, a right detection unit 92 connected to the first right electrode 12b, and a contact state determination unit 93 that determines whether all the electrodes are in a contact state based on outputs of the left detection unit 91 and the right detection unit 92.

The left detection unit 91 includes a left comparator 910, a left bias power source 911, a left switching element 912, a left pull-up resistance 913, a left RC filter 914, a left reference voltage power source 915, left reference voltage resistances 916a, 916b, and left hysteresis resistances 917a, 917b.

The left bias power source 911 applies a bias voltage (for example, about 3 V) to the left electrode 12a so that the left electrode 12a has a higher bias potential than the second right electrode 12c. The left switching element 912 is configured by a field effect transistor (FET), etc., for example, and turns ON/OFF the left bias power source 911 and the circuit under the control of the control unit 101. The left pull-up resistance 913 maintains the potential of the connected circuit at a high potential, and the left RC filter 914 removes a high-frequency component and inputs the voltage from the left bias power source 911 to the negative input terminal of the left comparator 910. Hereinafter, the potential input to the negative input terminal of the left comparator 910 is referred to as the left bias potential.

A predetermined contact detection reference voltage (for example, about 1.5 V) supplied from the left reference voltage power source 915 and adjusted by the left reference voltage resistances 916a, 916b is input to the positive input terminal of the left comparator 910. Hereinafter, the potential input to the positive input terminal of the left comparator 910 is referred to as the left detection reference potential.

The left comparator 910 is configured by, for example, an operational amplifier, and outputs “High” when the left bias potential decreases by a predetermined hysteresis amount with respect to the left detection reference potential. On the other hand, when the left bias potential is equal to or higher than the left detection reference potential, “Low” is output.

When both the left electrode 12a and the second right electrode 12c are correctly in contact with the skin of the body, a current flows through the impedance of the human body to the second right electrode 12c having a lower potential than the left electrode 12a, a voltage drop occurs in the left pull-up resistance 913, and the left bias potential decreases. Thus, the output of the left comparator 910 changes from “Low” to “High”. Note that the circuit of the dashed line portion in the drawing indicates the path of the current via the impedance of the human body.

Similar to the left detection unit 91, the right detection unit 92 includes a right comparator 920, a right bias power source 921, a right switching element 922, a right pull-up resistance 923, a right RC filter 924, a right reference voltage power source 925, right reference voltage resistances 926a, 926b, and right hysteresis resistances 927a, 927b.

The right bias power source 921 applies a bias voltage to the first right electrode 12b so that the first right electrode 12b has a higher bias potential than the second right electrode 12c. Other configurations and functions of the respective elements of the right detection unit 92 are the same as those of the left detection unit 91 with respect to the left electrode 12a, and thus detailed description thereof will be omitted.

The contact state determination unit 93 is configured by, for example, an AND circuit, and when both of the left comparator 910 and the right comparator 920 output “High”, the contact state determination unit 93 determines that all the electrodes of the left electrode 12a, the first right electrode 12b, and the second right electrode 12c are correctly in contact with each other, and outputs the determination result to the control unit 101.

Note that, as illustrated in FIG. 3, the left electrode 12a is connected to the positive input terminal of the differential amplifier 94, and the first right electrode 12b is connected to the negative input terminal of the differential amplifier 94, and the second right electrode 12c is connected to GND. The differential amplifier 94 amplifies and outputs the potential difference between the left electrode 12a and the first right electrode 12b, and the output is transmitted via a filter circuit (not illustrated) to the amplifier unit 102, the AD conversion unit 103, to perform the electrocardiographic measurement.

### Electrocardiographic Measurement Process Using Portable Electrocardiograph

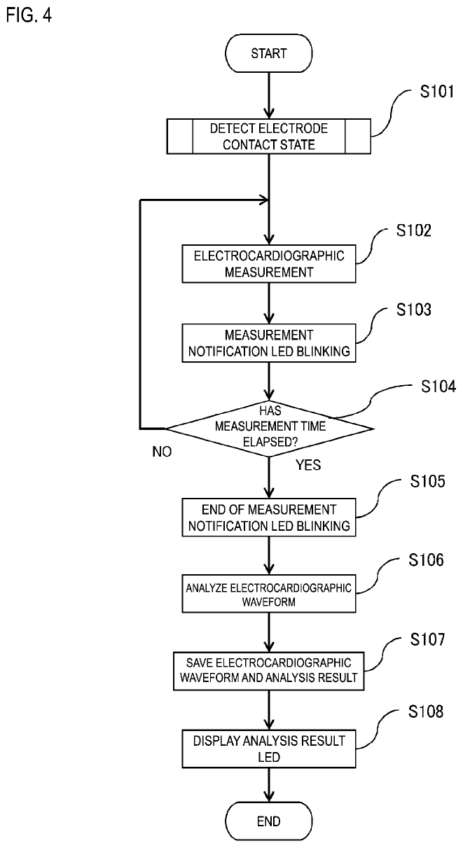

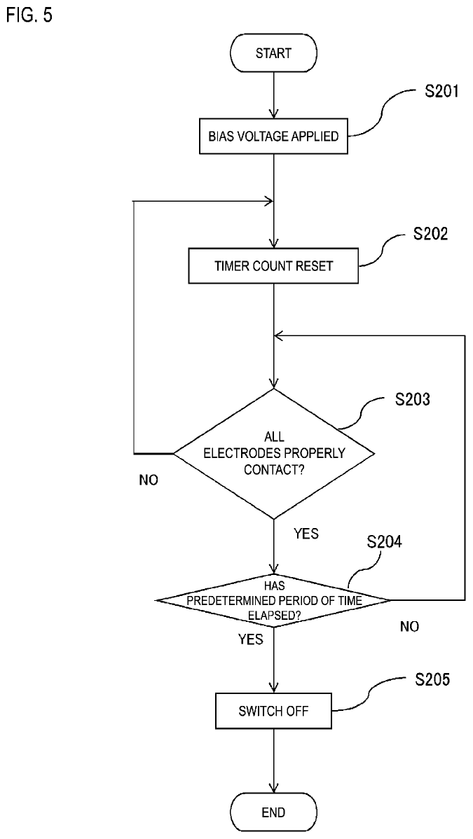

Now, based on FIGS. 1 to 5, a description will be given for operation of the portable electrocardiograph 10 when the electrocardiographic measurement is performed. FIG. 4 is a flowchart illustrating a procedure of processing when performing electrocardiographic measurement using the portable electrocardiograph 10, and FIG. 5 is a flowchart illustrating a subroutine for performing electrode contact detection processing in the portable electrocardiograph 10.

Referring to FIG. 4, prior to measurement, the user operates the power switch 16 to turn ON the power source of the portable electrocardiograph 10. As a result, the power source LED 16a is turned on to indicate that the power source is ON. Then, the user holds the portable electrocardiograph 10 with the right hand, with the right-hand index finger in contact with the first right electrode 12b and the second right electrode 12c, and with the left electrode 12a in contact with the skin at a location to be measured. Then, the control unit 101 detects the contact state of each of the electrodes via the electrode unit 12 and the contact state detection unit 111 (S101).

Here, the processing of the subroutine of step S101 will be described with reference to FIG. 5. First, when the power switch 16 is turned ON, the control unit 101 turns ON the left switching element 912 and the right switching element 922, and applies a bias voltage to the left electrode 12a and the first right electrode 12b (S201).

As described above, when all of the left electrode 12a, the first right electrode 12b, and the second right electrode 12c are in contact with the body, both the left comparator 910 and the right comparator 920 output “High”, and the contact state determination unit 93 outputs the result to the control unit 101. Then, if the “High” signal is continuously output for a predetermined time (for example, 3 seconds), it is assumed that each electrode is correctly in contact with the measurement target. Here, whether or not the predetermined time has elapsed may be determined by referring to the timer unit 104. In step S202, the control unit 101 resets (sets to 0) a timer count value (hereinafter referred to as a contact time count value) for measuring a time during which all the electrodes are in the contact state.

Next, in step S203, when it is determined that each of the left electrode 12a, the first right electrode 12b, and the second right electrode 12c is in contact with the body, the control unit 101 proceeds to step S204 and determines whether or not a predetermined time has elapsed in that state. On the other hand, when it is determined in step S203 that all the electrodes are not correctly contacted, the process returns to step S202, the contact time count value is reset, and the subsequent processing is repeated.

When it is determined in step S204 that the predetermined time has not elapsed, the process returns to step S203 and the subsequent processes are repeated. On the other hand, when it is determined in step S204 that the predetermined time has elapsed, the left switching element 912 and the right switching element 922 are turned OFF to invalidate the pull-up resistance (step S205), and the subroutine is ended.

Returning to the explanation of FIG. 4, after the subroutine of step S101 ends, the control unit 101 executes the actual electrocardiographic measurement process (step S102). While the electrocardiographic measurement is performed, the control unit 101 stores the measurement value in the storage unit 105 at any time, and displays that the electrocardiographic measurement is being performed by blinking the measurement state notification LED 13 on the front surface of the main body at a predetermined rhythm (S103).

Then, the control unit 101 performs processing for determining whether the elapsed time of the electrocardiographic measurement has reached a predetermined measurement time (for example, 30 seconds) (step S104). Here, if it is determined that the predetermined amount of time has not elapsed, the process returns to step S102, and the subsequent processing is repeated. On the other hand, if it is determined that the predetermined measurement time has elapsed, the measurement is ended, and a process of terminating the blink of the measurement state notification LED 13 is performed (step S105).

Next, the analysis unit 110 of the control unit 101 performs analysis of the measured data (electrocardiographic waveform) stored in the storage unit 105 (S106), and the analysis result is stored in a long term storage device along with the electrocardiographic waveform (S107). Then, the control unit 101 displays the result of the analysis by the analysis result notification LED 14 (S108), and ends the series of processes. Note that for the display of the analysis result, for example, the LED may be lighted only in a case where the electrocardiographic waveform is found abnormal or may be lighted in accordance with a lighting and blinking method corresponding to the analysis result.

According to the portable electrocardiograph 10 of the present embodiment having the above-described configuration, the user can start the measurement without performing operation other than bringing the electrodes into contact with the measurement site after operating the power switch 16, and since the measurement is not started unless all the electrodes are appropriately brought into contact, a highly accurate measurement result can be obtained.

In addition, since the first right electrode 12b is connected to GND and functions as the GND electrode, the GND can be used in common with an AD conversion unit of signals, and it is easy to remove an in-phase noise of signals at the time of AD conversion.

### Modification Example

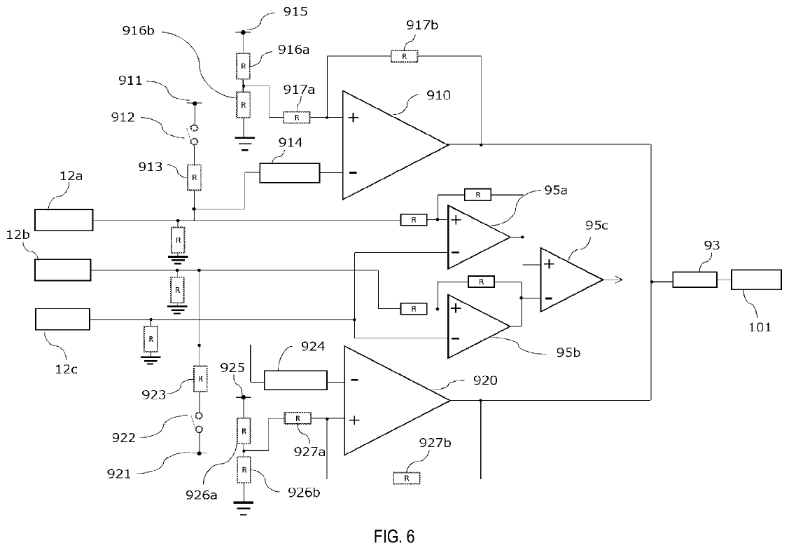

In the above embodiment, the first right electrode 12b functions as a GND electrode, but such a configuration is not necessarily required. FIG. 6 illustrates another configuration example of the portable electrocardiograph. The same components as those in the first embodiment are denoted by the same reference numerals, and detailed description thereof will be omitted.

As illustrated in FIG. 6, the portable electrocardiograph according to the present modification example includes three differential amplifiers, i.e., a left differential amplifier 95a, a right differential amplifier 95b, and a left-right differential amplifier 95c, and is configured to measure an electrocardiographic waveform based on outputs of the differential amplifiers.

To be more specific, the potential of the left electrode 12a is input to the positive side input of the left differential amplifier 95a, the potential of the second right electrode 12c is input to the negative side input thereof, and the potential difference therebetween is output. Additionally, the potential of the first right electrode 12b is input to the positive side input of the right differential amplifier 95b, the potential of the second right electrode 12c is input to the negative side input thereof, and the potential difference therebetween is output.

Also, the output potential of the left differential amplifier 95a is input to the positive side input of the left-right differential amplifier 95c, and the output potential of the right differential amplifier 95b is input to the negative side input, and the potential difference therebetween is output. Then, the signal output from the left-right differential amplifier 95c is transmitted to the amplifier unit 102 and the AD conversion unit 103 via a filter circuit (not illustrated), whereby the electrocardiographic measurement is performed.

With such a configuration, since a signal is obtained by amplifying a potential difference between the left electrode 12a and the first right electrode 12b using the second right electrode 12c as a reference electrode, the in-phase noise can be easily removed when the signal is amplified.

### Other Points

The description of the embodiment described above is merely illustrative of the present invention, and the present invention is not limited to the specific embodiments described above. Within the scope of the technical idea of the present invention, various modifications and combinations may be made.

For example, the switching element in the above embodiment is not limited to the FET, and the comparator and the differential amplifier do not necessarily have to be an operational amplifier. Although not described in detail in the above embodiment, the electrocardiograph and another information terminal device can be used in cooperation with each other by the BLE communication function of the communication unit 109. Conversely, an electrocardiograph that does not include a communication function and an LED display unit can be used.

Although the present invention is applied to a portable electrocardiograph in the above description, the present invention can also be applied to a non-portable electrocardiograph, and can also be applied to other biological measurement devices such as a body composition meter.

### REFERENCE NUMERALS LIST

10 Portable electrocardiograph
12a Left electrode
12b First right electrode
12c Second right electrode
13 Measurement state notification LED
14 Analysis result notification LED
15 Battery cover
16 Power switch
16a Power source LED
17 Communication button
17a BLE Communication LED
18 Available memory display LED
19 Battery change LED
91 Left detection unit
92 Right detection unit
93 Contact state determination unit
94 Differential amplifier
95a Left differential amplifier
95b Right differential amplifier
95c Left-right differential amplifier
910 Left comparator
911 Left bias power source
912 Left switching element
913 Left pull-up resistance
914 Left RC filter
915 Left reference voltage power source
916a, 916b Left reference voltage resistance
917a, 917b Left hysteresis resistance

1. A biological information measurement device including a first electrode, a second electrode, and a third electrode, the biological information measurement device measuring biological information of a measurement target based on a potential difference between the first electrode and the second electrode, the biological information measurement device comprising:
an electrode contact detection means configured to detect and output a state in which all of the first electrode, the second electrode, and the third electrode are in contact with a surface of the measurement target; and
a control means configured to execute a measurement process of measuring the biological information, wherein
the electrode contact detection means includes:
a bias power source configured to apply a voltage to each of the first electrode and the second electrode so that the first electrode and the second electrode have a contact detection potential higher than a potential of the third electrode;
a first comparator and a second comparator connected to the first electrode and the second electrode, respectively, and configured to compare the contact detection potential with respective potentials of the first electrode and the second electrode; and
a contact state determination unit configured to determine whether or not all of the first electrode, the second electrode, and the third electrode are in contact with the surface of the measurement target based on outputs of the first comparator and the second comparator, and
the control means is configured to execute a process of opening the first electrode, the second electrode, and the bias power source, and execute the measurement process when the electrode contact detection means outputs that all of the first electrode, the second electrode, and the third electrode are in contact with the surface of the measurement target.

2. The biological information measurement device according to claim 1, wherein
the third electrode is a ground electrode,
the biological information measurement device includes a first differential amplifier connected to the first electrode and the second electrode, the first differential amplifier being configured to amplify and output a potential difference between the first electrode and the second electrode, and
the control means is configured to measure the biological information of the measurement target based on an output of the first differential amplifier.

3. The biological information measurement device according to claim 1, comprising:
a second differential amplifier connected to the first electrode and the third electrode, and configured to amplify and output a potential difference between the first electrode and the third electrode;
a third differential amplifier connected to the second electrode and the third electrode, and configured to amplify and output a potential difference between the second electrode and the third electrode; and
a fourth differential amplifier connected to output sides of the second differential amplifier and the third differential amplifier, and configured to amplify and output a potential difference between an output voltage of the second differential amplifier and an output voltage of the third differential amplifier, wherein
the control means is configured to measure the biological information of the measurement target based on an output of the fourth differential amplifier.

4. The biological information measurement device according to claim 1, wherein
the biological information is an electrocardiographic waveform.

5. The biological information measurement device according to claim 1, wherein
the biological information measurement device is a portable device.

6. The biological information measurement device according to claim 2, wherein
the biological information is an electrocardiographic waveform.

7. The biological information measurement device according to claim 3, wherein
the biological information is an electrocardiographic waveform.

8. The biological information measurement device according to claim 2, wherein
the biological information measurement device is a portable device.

9. The biological information measurement device according to claim 3, wherein
the biological information measurement device is a portable device.

10. The biological information measurement device according to claim 4, wherein
the biological information measurement device is a portable device.

11. The biological information measurement device according to claim 6, wherein
the biological information measurement device is a portable device.

12. The biological information measurement device according to claim 7, wherein
the biological information measurement device is a portable device.
